(19) United States
(12) Patent Application Publication
(10) Pub. No.: US 2012/0232003 A1
(43) Pub. Date: Sep. 13, 2012

(54) COMPOSITIONS AND METHODS FOR DIABETES TREATMENT

(76) Inventors: Joseph S. Takahashi, Dallas, TX (US); Steve A. Kay, San Diego, CA (US)

(21) Appl. No.: 13/255,641

(22) PCT Filed: Mar. 15, 2010
(86) PCT No.: PCT/US10/00783

§ 371 Date: Apr. 20, 2012

Related U.S. Application Data
(60) Provisional application No. 61/160,160, filed on Mar. 13, 2009.

(51) Int. Cl. A61K 31/4045; A61K 31/4985; A61P 3/06; A61K 38/26; A61P 3/10; A61K 38/22; C07D 209/14

(52) U.S. Cl. 514/7.2; 514/415; 514/6.9; 514/249; 548/507

(57) ABSTRACT

The present invention relates to methods and compositions for the treatment or prevention of type 2 diabetes by administering an effective amount of a melatonin receptor agonist to a human subject in need of such treatment or prevention.

### FIELD OF THE INVENTION

The present invention relates to compositions and methods for the treatment or prevention of diabetes and insulin resistance using melatonin and melatonin receptor agonist compounds.

### BACKGROUND OF THE INVENTION

Diabetes mellitus afflicts an estimated 23.6 million people in the U.S., or about 7.8% of the population, and is the seventh leading cause of death. Diabetes lowers average life expectancy by up to 15 years, increases cardiovascular disease risk two- to four-fold, and is the main cause of kidney failure, lower limb amputations, and adult-onset blindness. Type 2 diabetes accounts for up to 95 percent of diabetes cases. Rates of diabetes are expected to rise substantially as the U.S. population ages and becomes increasingly overweight, sedentary, and ethnically diverse.

In normal subjects, after an overnight fast, glucose is produced from hepatic glycogen (25%) and gluconeogenesis (75%). Gluconeogenesis occurs in both the kidney and the liver.

Catecholamines and glucagon, via a cAMP-dependent signaling pathway, stimulate gluconeogenesis. Following a meal, there is an increase in secretion of the incretin hormone, glucagon-like peptide 1 (GLP-1), from enteroendocrine L-cells. GLP-1 binds to its receptor on pancreatic beta cells, inducing activation of the cAMP-generating enzyme, adenylyl cyclase. As a result, cAMP levels increase, promoting the cellular responses to increased blood glucose levels, which include insulin secretion and insulin gene expression. Insulin, in turn, inhibits gluconeogenesis and promotes the cellular uptake of glucose from the blood. Diabetes is a state of chronic hyperglycemia, meaning that blood glucose levels are chronically elevated. Although circulating insulin levels are also frequently elevated early in type 2 diabetes, a deficiency of intracellular insulin and increased cellular resistance to many of insulin's actions simultaneously occur. Insulin resistance leads to decreased glucose uptake by muscle and liver cells and inappropriate hepatic glucose production and lipolysis in adipose tissue, both of which are normally suppressed by insulin.

Melatonin (N-acetyl-5-methoxyindole-3-ethaneamine), an indole derivative, is a hormone produced from the amino acid tryptophan and excreted by the pineal gland. It is well-known as a regulator of circadian rhythms and the sleep-wake cycle of vertebrates via its effects on the master circadian pacemaker, the suprachiasmatic nucleus (SCN). In the SCN, melatonin affects the amplitude of the circadian signal and the phase shift of circadian rhythms of neuronal firing, which may be related to sleep timing. Melatonin is produced rhythmically on an intrinsic cycle of approximately 24-hours such that plasma levels are low in the daytime and higher at night. Two melatonin receptors have been identified, MT₁ and MT₂ (also referred to as MTNR1A and MTNR1B). Each activates inhibitory G-protein coupled receptors (Gᵢ). Receptor activation initiates multiple signaling pathways, most commonly the inhibition of cAMP formation.

Melatonin plays a direct role in the regulation of insulin secretion via a complex pattern of intracellular signaling pathways in pancreatic beta cells and diabetes is associated with a reduction in melatonin secretion (see review by Peschke, J. Pineal Res. (2008) 44:26-40). The melatonin signaling pathways include at least three parallel pathways mediated by cAMP, cGMP, and IP₃. The predominant pathway is believed to be the cAMP pathway, which results in an inhibition of insulin secretion. Co-administration of melatonin and glucagon-like peptide 1 (GLP-1) inhibits GLP-1 induced insulin secretion. The cGMP pathway also leads to inhibition of insulin secretion. In contrast, the melatonin-induced release of IP₃ leads to insulin release. This is consistent with the observation that melatonin stimulates glucose transport in mouse C2C12 skeletal muscle cells (Ha et al., J. Pineal Res. (2006) 41:67-72)

Melatonin has also been shown to sensitize pancreatic beta cells to cAMP signaling in experiments using rat insulinoma cells (INS1) (Kemp et al.; Mol. Cell. Endocrinol. (2002) 191:157-166). In these cells, an overnight pretreatment with melatonin resulted in a marked increase in insulin secretion, cAMP-response element-mediated gene expression, and insulin-promoter-driven luciferase gene expression in response to GLP-1 or forskolin. In contrast, melatonin antagonized GLP-1 induced gene expression and insulin secretion when the cells were exposed to both melatonin and GLP-1 at the same time.

In addition to its activity as a regulator of circadian rhythms and glucose homeostasis, melatonin is also a potent antioxidant. The diabetic state enhances oxidative stress caused by the generation of free radicals and there is evidence that this stress can be reduced by melatonin. For example, melatonin provided limited protection against hyperglycemia-induced oxidative damage in a rat model of diabetes, without affecting blood glucose levels (Paskaloglu et al., Eur. J. Pharmacol. (2004) 499:345-354). In humans, the combination of melatonin and the antioxidant zinc acetate, used alone or in combination with metformin, improved impaired fasting and post-prandial glycemic control in patients with type 2 diabetes (Hussain et al., Saudi Med. J. (2006) 27:1483-1488; Kadhim et al., J. Pineal Res. (2006) 41:189-193). This treatment also decreased the level of glycated hemoglobin and improved diabetes-related complications such as impaired lipid profiles and microalbuminuria in type 2 diabetes. Due to its potent antioxidant activity, melatonin has been suggested as an adjunct to sulfonylurea and metformin therapy for progressive insulin resistance and type 2 diabetes (see U.S. Pat. No. 7,060,295). However, others have implicated melatonin in the pathogenesis of diabetes and suggested that therapy for type 2 diabetes should include inhibition of melatonin (see Lyssenko et al., Nature Genetics (2009) 41:82-88); Staiger et al., PlosOne December 2008, 3:e3962). This suggestion was based on the association of a common variant of the melatonin receptor with an increased risk of type 2 diabetes and impaired insulin secretion from pancreatic beta cells.

### SUMMARY OF THE INVENTION

The present invention provides methods and compositions for the treatment or prevention of diabetes, specifically type 2 diabetes and insulin resistance by administering a sustained, continuous dose of a melatonin receptor (MTR) agonist to a human subject in need thereof.

A subject in need of treatment is one who has been diagnosed with type 2 diabetes. A subject in need of prevention is one who shows one or more clinical indicators of progression toward type 2 diabetes such as insulin resistance, impaired glucose tolerance, or elevated fasting blood glucose levels. Other clinical indicators of progression toward type 2 diabetes include, but are not limited to, hyperglycemia induced by or associated with tube-feeding during hospital treatments, and shift-work/jet-lag induced impaired glucose tolerance.

Preferably, the MTR agonist is administered from about 6 to 12 hours prior to a meal. In one aspect of the invention, the long and continuous treatment with an MTR agonist that has central effects and that promotes sleep is believed to have beneficial effects on glucose metabolism. In one embodiment, the MTR agonist is continuously administered as a sustained release formulation for a period of time up to about 12 hours, preferably for about 6 hours to about 12 hours. In a preferred embodiment, the MTR agonist is a molecule that does not cross the blood-brain barrier and is active only in the periphery or the MTR agonist is formulated so that it does not cross the blood-brain barrier and is active only in the periphery.

In specific embodiments, the MTR agonist is administered in combination with one or more anti-diabetic agents, preferably in a controlled release formulation as described herein. The methods and compositions of this invention advantageously improve the therapeutic index and improve the therapeutic response to anti-diabetic agents, and facilitate administration of lower amounts of anti-diabetic agents (than would be used absent the administration of the MTR agonists according to this invention). This in turn is expected to reduce the side effects and complications associated with such drugs (such as morbidity and mortality resulting from hypoglycemia and hyperinsulinemia, including, inter alia, fluid retention and weight gain).

The invention provides a method for the treatment of type 2 diabetes in a human subject in need thereof, comprising administering to the subject an effective amount of a melatonin receptor (MTR) agonist about 6-12 hours before a meal. The MTR agonist can be melatonin. In one embodiment, the MTR agonist is administered by itself (“monotherapy”). In one embodiment, the MTR agonist is administered as part of a combination therapy with one or more therapeutic agents. In one embodiment, the one or more therapeutic agents is selected from the group consisting of a biguanide, glucagon-like peptide 1, a glucagon-like peptide 1 receptor activator, a dipeptidyl peptidase 4 inhibitor, an insulin sensitizer, and a hypolipidemic agent. In one embodiment, the one or more therapeutic agents is selected from the group consisting of a dipeptidyl peptidase 4 inhibitor, an insulin sensitizer, and a hypolipidemic agent. In one embodiment, the one or more therapeutic agents is selected from the group consisting of an activator of gastric inhibitory peptide receptor, a glucokinase activator, a ghrelin receptor agonist, an orexin receptor agonist, an oxyntomodulin receptor agonist, a G-protein coupled receptor 40 or 119 agonist, glucose-dependent insulinotropic peptide, an agonist of an RGS protein, and an insulin secretagogue (such as, e.g., a glinide or a sulfonylurea).

In one embodiment, the method comprises an MTR agonist formulated for oral administration. In another embodiment, the method comprises an MTR agonist formulated as a sustained release formulation that releases the MTR agonist for a period of time of up to about 12 hours. Preferably, the sustained release formulation releases the MTR agonist for a period of time of about 6 hours to about 12 hours.

The invention provides a pharmaceutical composition comprising a melatonin receptor (MTR) agonist for the treatment of diabetes, wherein the composition is formulated to provide for sustained or controlled release of the MTR agonist when administered as monotherapy. The sustained release can be for a continuous period of time up to about 12 hours, preferably for about 6 hours to about 12 hours.

The invention also provides a pharmaceutical composition comprising a melatonin receptor (MTR) agonist and one or more therapeutic agents for the treatment of diabetes, wherein the composition is formulated to provide for the immediate release of the MTR agonist and the delayed release of the one or more therapeutic agents. In one embodiment, the release of the one or more therapeutic agents is delayed for about 6 to 12 hours. In one embodiment, the one or more therapeutic agents is selected from glucagon-like peptide 1, a glucagon-like peptide 1 receptor activator, gastric inhibitory peptide, a gastric inhibitory peptide receptor activator, glucose-dependent insulinotropic peptide, a glucokinase activator, a ghrelin receptor agonist, an insulin secretagogue, an orexin receptor agonist, an oxyntomodulin receptor agonist, a G-protein coupled receptor 40 or 119 agonist, an agonist of an RGS protein, a dipeptidyl peptidase 4 inhibitor, a biguanide, an insulin sensitizer, and a hypolipidemic agent.

In another aspect of the invention, a pharmaceutical composition comprising a melatonin receptor (MTR) agonist and one or more therapeutic agents for the treatment of diabetes is provided, wherein the composition is formulated as a controlled (or “sustained”) release formulation to provide for the sustained release of the MTR agonist and the immediate release of the one or more additional therapeutic agents. The sustained release of the MTR agonist can be for a continuous period of time up to about 12 hours.

In a preferred embodiment, the one or more additional therapeutic agents can be selected from glucagon-like peptide 1, gastric inhibitory peptide, a gastric inhibitory peptide receptor activator, a GLP-1 receptor activator, glucose-dependent insulinotropic peptide, a glucokinase activator, a ghrelin receptor agonist, an orexin receptor agonist, an oxyntomodulin receptor agonist, a G-protein coupled receptor 40 or 119 agonist, an agonist of an RGS protein, a biguanide, an insulin sensitizer, and a hypolipidemic agent.

In other aspects of the invention, a pharmaceutical composition comprising a melatonin (MTR) agonist and one or more therapeutic agents for the treatment of diabetes is provided, wherein the composition is formulated as a controlled (or “sustained”) release formulation to provide for the sustained release of the MTR agonist and the delayed release of the one or more additional therapeutic agents. The delayed release of the one or more additional therapeutic agents can be right before a meal and after the sustained release of the MTR agonist.

In a preferred embodiment, the one or more additional therapeutic agents comprises a dipeptidyl peptidase 4 inhibitor, preferably sitagliptin.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and are encompassed by the following detailed description and claims.

### BRIEF DESCRIPTION OF THE DRAWINGS

FIGS. 1A-1D depicts graphs measuring the blood glucose and insulin levels of mice receiving a 12-hour sustained dose of melatonin, a 1-hour sustained dose of melatonin, or vehicle alone.

FIGS. 2A and 2B depict graphs measuring the body weight and blood glucose levels of mice receiving vehicle alone, oral dosing vehicle alone, vehicle and oral dosing vehicle together, 12-hour sustained melatonin alone, sitagliptin alone, the combination of 12-hour sustained melatonin and sitagliptin, and mice receiving no treatment.

### DETAILED DESCRIPTION OF THE INVENTION

The present invention provides methods and compositions for the treatment or prevention of diabetes, specifically type 2 diabetes, in a human subject in need thereof by administering a melatonin receptor (MTR) agonist to the subject. Preferably, the MTR agonist is administered from about 6 to 12 hours prior to a meal. More preferably, the MTR agonist is administered prior to a normal sleep cycle (at a time when the patient's biological clock operates as if it is nighttime). In other embodiments, the MTR agonist is formulated as a sustained or controlled release formulation that releases the MTR agonist for a continuous period of time of up to about 12 hours.

In one embodiment, the MTR agonist is formulated with one or more additional therapeutic agents for the treatment of diabetes. Preferably, the formulation provides for the immediate release of the MTR agonist and for the delayed release of the one or more additional therapeutic agents. In certain embodiments, the release of the one or more additional therapeutic agents is delayed for about 2 hours, 4 hours, 6 hours, 8 hours, 10 hours, or 12 hours following administration, preferably for about 6 to 12 hours. In a specific embodiment, the one or more additional therapeutic agents formulated for delayed release is selected from the group consisting of glucagon-like peptide 1 (GLP-1), a glucagon-like peptide 1 (GLP-1) receptor activator (e.g., exenatide or liraglutide, also known in the art as BYETTA and VICTOZA, respectively); gastric inhibitory peptide, a gastric inhibitory peptide receptor activator; glucose-dependent insulinotropic peptide; a glucokinase activator; a ghrelin receptor agonist; an orexin receptor agonist; an oxyntomodulin receptor agonist; a GPR40 or GPR119 receptor agonist; an agonist of an RGS protein; a dipeptidyl peptidase 4 inhibitor (e.g., sitagliptin, also known in the art as JANUVIA, and saxagliptin, known in the art as ONGLYZA); a biguanide (e.g., METFORMIN); a thiazolidinedione insulin sensitizer (or related PPAR gamma modulators); and a fibrate hypolipidemic agent (e.g., fenofibrate).

In other embodiments, the formulations of the invention (including pharmaceutical compositions of the invention) provide for the controlled or sustained release of the MTR agonist (which can be immediate or delayed relative to the one or more additional therapeutic agents) and immediate release or delayed release of the one or more additional therapeutic agents. The sustained release of the MTR agonist can be for a continuous period of time up to about 12 hours, i.e., 1 hour, 2 hours, 3 hours, 4 hours, 5 hours, 6 hours, 7 hours, 8 hours, 9 hours, 10 hours, 11 hours, 12 hours, or more, and including half-hour increments (i.e., 1.5 hours, 2.5 hours, and the like). Preferably, the sustained release of the MTR agonist is for a period of time of 6 hours to 12 hours. In some embodiments, the delayed release of the one or more additional therapeutic agents can be before a meal and after the sustained release of the MTR agonist, e.g., after 6 hours to 12 hours.

The term “MTR agonist” as used herein refers to any agonist of a melatonin receptor, specifically the MT1 or MT2 receptor. Exemplary MTR agonists include melatonin (N-acetyl-5-methoxyindole-3-ethaneamine) and biologically active derivatives thereof, including, without limitation, 5-methoxytryptamine, 5-methoxytryptophan, 5-methoxytryptophol, 5-methoxy-indole-3-acetic acid, and 6-hydroxymelatonin. Additional agonists include ramelteon (ROZEREM), agomelatine (VALDOXAN), and PD6735, and their biologically active derivatives.

In one embodiment, the MTR agonist is administered by itself as monotherapy. In other embodiments, the MTR agonist is administered in combination with one or more additional therapeutic agents. As used herein, the term “in combination with” when used in the context of administration means administration of the MTR agonist prior to, at substantially the same time as, or after, the one or more additional therapeutic agents. In certain embodiments, the MTR agonist is formulated with one or more additional therapeutic agents such that, although administered at the same time, the MTR agonist is released into the body prior to the release of the one or more therapeutic agents. In one embodiment, the MTR agonist acts as an adjunct to one or more therapeutic agents, meaning that the MTR agonist is added to an ongoing therapy comprising the one or more therapeutic agents. In certain embodiments, the MTR agonist enhances, increases, or potentiates the activity of the one or more therapeutic agents. In one embodiment, combination with the MTR agonist permits the administration of a lower dose of the one or more therapeutic agents. In another embodiment, one or more deleterious side effects of the one or more additional therapeutic agents is reduced when the agent(s) is used in combination with the MTR agonist.

In one embodiment, the one or more therapeutic agents is selected from the group consisting of a biguanide, a glucagon-like peptide 1 receptor activator, a dipeptidyl peptidase 4 inhibitor, an insulin sensitizer, and a hypolipidemic agent. In a particular embodiment, the one or more therapeutic agents is selected from the group consisting of a dipeptidyl peptidase 4 inhibitor, an insulin sensitizer, and a hypolipidemic agent.

In another embodiment, the one or more therapeutic agents is selected from the group consisting of a gastric inhibitory peptide receptor activator, a glucokinase activator, a ghrelin receptor agonist, an orexin receptor agonist, an oxyntomodulin receptor agonist, a G-protein coupled receptor 40 or 119 agonist, glucose-dependent insulinotropic peptide, an agonist of an RGS protein, and an insulin secretagogue.

In one embodiment, the one or more therapeutic agents does not include zinc, a sulfonylurea, a biguanide, GLP-1, or a GLP-1 receptor activator.

In one embodiment, the MTR agonist is not administered in combination with an antioxidant.

In another embodiment, the MTR agonist is administered in combination with an agent selected from the group consisting of an inhibitor of dipeptidyl peptidase 4 (DPP4), glucose-dependent insulinotropic peptide, an agonist of a G-protein coupled receptor, preferably GPR40, and an agonist of an RGS protein. Preferably, the DPP4 inhibitor is sitagliptin.

The term “treating” or “treatment” as used herein refers to a reduction, a partial improvement, amelioration, or a mitigation of at least one clinical symptom associated with the type 2 diabetes being treated, such as, e.g., an increase in insulin levels and/or a decrease in blood glucose levels. Measurement of at least one clinical symptom associated with type 2 diabetes can include methods known in the art, such as, for example, by measurement of insulin levels. One particular method is by measurement of blood glucose levels by oral glucose tolerance test.

The term “preventing” or “prevention” refers to inhibition or delay in the onset or progression of at least one clinical symptom associated with type 2 diabetes. The term “effective amount” refers to an amount that provides some improvement or benefit to the subject. In certain embodiments, an “effective amount” is an amount that provides some alleviation, mitigation, and/or decrease in at least one clinical symptom of the type 2 diabetes to be treated. In other embodiments, the “effective amount” is the amount that provides some inhibition or delay in the onset or progression of at least one clinical symptom associated with type 2 diabetes. The therapeutic effects need not be complete or curative, as long as some benefit is provided to the subject. The term “subject” preferably refers to a human subject but may also refer to a non-human primate or other mammal preferably selected from among a mouse, a rat, a dog, a eat, a cow, a horse, or a pig.

The terms “intermittent” or “intermittently” as used herein means stopping and starting at either regular or irregular intervals. Also used in this regard is the term “sustained” or “continuous”, which means maintaining for a period of time without interruption. Sustained or continuous administration can be achieved by, for example, a controlled release or sustained release formulation.

This invention, in addition to the above listed compounds, is intended to encompass the use of homologs and analogs of such compounds. In this context, homologs are molecules having substantial structural similarities to the above-described compounds and analogs are molecules having substantial biological similarities regardless of structural similarities.

1.1 Formulations

The MTR agonists for use in the methods of the invention are formulated to provide maximum efficacy for the intended use. The MTR agonists can be a component of a pharmaceutical composition for the treatment of diabetes, wherein the composition is formulated to provide for sustained or controlled release of the MTR agonist. The sustained release can be for a period of time up to about 12 hours.

In a preferred embodiment, the MTR agonist is a molecule that does not cross the blood-brain harrier and is active only in the periphery or the MTR agonist is formulated so that it does not cross the blood-brain barrier and is active only in the periphery. In another preferred embodiment, the MTR agonist is a molecule that does cross the blood-brain barrier or the MTR agonist is formulated so that it does cross the blood-brain barrier.

In one embodiment, the MTR agonist is formulated for oral administration and preferably is formulated as a solid dosage for oral administration. In other embodiments, the MTR agonist is formulated as a sustained release formulation that can administer the MTR agonist for a period of time of up to about 12 hours, preferably about 6 hours to about 12 hours.

In one embodiment, the MTR agonist is formulated with one or more additional therapeutic agents in a controlled (time-dependent) release formulation. Controlled release in this context includes delayed sustained release, delayed controlled release, delayed slow release, delayed prolonged release, delayed extended release, and a sudden release or “burst.” According to this embodiment, the formulation provides for the release of the MTR agonist prior to the release of the one or more additional therapeutic agents. Preferably, the MTR agonist is substantially released at least several hours, preferably 2 to 12 hours, before the release of the one or more additional therapeutic agents. In one embodiment, the MTR agonist is released immediately following administration, and is administered for a sustained, continuous period of time of up to about 12 hours, preferably 6 hours to about 12 hours and the one or more additional therapeutic agents is released 2, 4, 6, 8, 10, or 12 hours after administration. Controlled release formulations are described in more detail in Section 1.1.1 below.

In other embodiments, the MTR agonist is substantially released for a period of time up to about 12 hours, i.e., 1 hour, 2 hours, 3 hours, 4 hours, 5 hours, 6 hours, 7 hours, 8 hours, 9 hours, 10 hours, 11 hours, 12 hours or more, and including half-hour increments (i.e., 1.5 hours, 2.5 hours, and the like). Preferably, the sustained release is for 6 hours to 12 hours. In this embodiment, the one or more additional therapeutic agents can be released immediately or its release can be delayed. The one or more additional therapeutic agent can be administered, for example, before a meal and after the sustained release of the MTR agonist of 6 hours to 12 hours.

In certain embodiments, the formulations comprise one or more pharmaceutically acceptable excipients. The term excipient as used herein broadly refers to a biologically inactive substance used in combination with the active agents of the formulation. An excipient can be used, for example, as a solubilizing agent, a stabilizing agent, a diluent, an inert carrier, a preservative, a binder, a disintegrant, a coating agent, a flavoring agent, or a coloring agent. Preferably, at least one excipient is chosen to provide one or more beneficial physical properties to the formulation, such as increased stability and/or solubility of the active agent(s). An MTR agonist as described herein is the primary active agent in the formulations of the present invention. However, the MTR agonist can be formulated with other active agents as described herein.

A “pharmaceutically acceptable” excipient is one that has been approved by a state or federal regulatory agency for use in animals, and preferably for use in humans, or is listed in the U.S. Pharmacopia, the European Pharmacopia or another generally recognized pharmacopia for use in animals, and preferably for use in humans.

Examples of excipients include certain inert proteins such as albumins; hydrophilic polymers such as polyvinylpyrrolidone; amino acids such as aspartic acid (which may alternatively be referred to as aspartate), glutamic acid (which may alternatively be referred to as glutamate), lysine, arginine, glycine, and histidine; fatty acids and phospholipids such as alkyl sulfonates and caprylate; surfactants such as sodium dodecyl sulphate and polysorbate; nonionic surfactants such as such as TWEEN®, PLURONICS®, or polyethylene glycol (PEG); carbohydrates such as glucose, sucrose, mannose, maltose, trehalose, and dextrins, including cyclodextrins; polyols such as mannitol and sorbitol; chelating agents such as EDTA; and salt-forming counter-ions such as sodium.

The formulations of the present invention may also contain pharmaceutically acceptable salts, buffering agents, or preservatives. Examples of such salts include those prepared from the following acids: hydrochloric, hydrobromic, sulfuric, nitric, phosphoric, maleic, acetic, salicylic, citric, boric, formic, malonic, succinic, and the like. Such salts can also be prepared as alkaline metal or alkaline earth salts, such as sodium, potassium or calcium salts. Examples of buffering agents include phosphate, citrate, acetate, and 2-(N-morpholino)ethanesulfonic acid (MES). Examples of preservatives include antioxidants such as vitamin A, vitamin E, vitamin C, retinyl palmitate, and selenium; the amino acids cysteine and methionine; citric acid and sodium citrate; and synthetic preservatives such as thimerosal, and alkyl parabens, including for example, methyl paraben and propyl paraben. Other preservatives include octadecyldimethylbenzyl ammonium chloride, hexamethonium chloride, benzalkonium chloride, benzethonium chloride, phenol, butyl or benzyl alcohol, chlorobutanol, catechol, resorcinol, cyclohexanol, 3-pentanol, and m-cresol.

In certain embodiments, the formulations of the invention may be prepared as a liquid or in a solid form such as a powder, tablet, pill or capsule. Liquid formulations may take such forms as suspensions, solutions or emulsions in oily or aqueous vehicles, and may contain formulatory agents such as suspending, stabilizing and/or dispersing agents. In one embodiment, the formulation is an aqueous solution. In another embodiment, the final formulation is lyophilized. In other embodiments, the formulation comprises a colloidal drug delivery system. Such drug delivery systems include, for example, liposomes, albumin microspheres, microemulsions, nano-particles and nanocapsules.

In one embodiment, the formulation is a liquid or lyophilized formulation suitable for injection in a mammal, preferably a human. In one embodiment, the formulation is sterile. In another embodiment, the formulation is a sterile lyophilized formulation which is suitable for injection upon reconstitution with an amount of an aqueous carrier. In one embodiment, the liquid or lyophilized formulation is prepared as a unit dosage form as described below. The formulations may or may not contain an added preservative.

In certain embodiments, the formulations further comprise one or more adjuvants. Examples of suitable adjuvants include potentiators of the immune response such as microbial derivatives (e.g., bacterial products, toxins such as cholera toxin and heat labile toxin from E. coli, lipids, lipoproteins, nucleic acids, peptidoglycans, carbohydrates, peptides), cells, cytokines, (e.g., dendritic cells, IL-12, and GM-CSF), hormones, and small molecules. Adjuvants contemplated include, but are not limited to, oil-based adjuvants (e.g., Freund's adjuvant), CpG oligonucleotides, aluminum salt adjuvants, calcium salt adjuvants, emulsions and surfactant-based formulations (e.g., MF59, ASO2, montanide, ISA-51, ISA-720, and QA21).

In embodiments where the formulation is an emulsion, suitable emulsifiers or emulsifying agents include any pharmaceutically acceptable emulsifier, preferably phospholipids extracted from egg yolk or soy bean, synthetic phosphatidyl cholines or purified phosphatidyl cholines from vegetable origin. Hydrogenated derivatives can also be used, such as phosphatidylcholine hydrogenated (egg) and phosphatidylcholine hydrogenated (soya). Emulsifiers may also be non-ionic surfactants such as poloxamers (for example Poloxamer 188 and 407), poloxamines, polyoxyethylene stearates, polyoxyethylene sorbitan fatty acid esters or sorbitan fatty acid esters. Ionic surfactants may also be used such as cholic acid and deoxycholic acid or surface active derivatives or salts thereof. The emulsifier can also be a mixture of one or more of the above ingredients. The emulsion may additionally contain other ingredients such as buffers, stabilizers and other lipids.

The formulations of the present invention can optionally be prepared as unit dosage forms. “Unit dosage form” refers to physically discrete units suitable for the intended use, i.e., as a single administration to the subject to be treated. Each unit contains a predetermined quantity of the active agent(s) formulated with the appropriate pharmaceutically acceptable excipient(s). Examples of unit dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non aqueous liquid suspensions, oil in water emulsions, or a water in oil liquid emulsions), solutions, and elixirs; liquid dosage forms suitable for subcutaneous administration to a subject; and sterile solids (e.g., crystalline or amorphous solids) that can be reconstituted to provide liquid dosage forms suitable for subcutaneous administration to a subject.

Additional information with regard to the methods of making the compositions and formulations and the ingredients comprising the compositions and formulations in accordance with the present invention can be found in standard references in the field, such as for example, “Remington's Pharmaceutical Sciences,” Mack Publishing Co., Easton, Pa.

1.1.1 Controlled Release/Sustained Release Formulations

In one embodiment, the “controlled release” (or “sustained release”) formulation comprises a slowly disintegrating core containing the one or more additional therapeutic agents surrounded by an outer layer. In some embodiments, the core optionally further comprises at least one of a lubricant, a flow promoting agent, a plasticizer, an anti-sticking agent, a surfactant, a wetting agent, a suspending agent, and a dispersing agent. In certain embodiments, the core also comprises a wicking agent such as silicon dioxide. The wicking agent may also be selected from a disintegrant such as microcrystalline cellulose to enhance the speed of water uptake. Other suitable wicking agents include, but are not limited to, kaolin, titanium dioxide, fumed silicon dioxide, alumina, niacinamide, sodium lauryl sulfate, low molecular weight polyvinyl pyrrolidone, m-pyrol, bentonite, magnesium aluminum silicate, polyester, polyethylene, and mixtures thereof. Non-limiting examples of a plasticizer include dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof. In one embodiment, the stiffening agent comprises cetyl alcohol. Non-limiting examples of wetting agents include a poloxamer, polyoxyethylene ethers, polyoxyethylene sorbitan fatty acid esters, polyoxymethylene stearate, sodium lauryl sulfate, sorbitan fatty acid esters, benzalkonium chloride, polyethoxylated castor oil, and docusate sodium. Non-limiting examples of suspending agents include alginic acid, bentonite, carbomer, carboxymethylcellulose, carboxymethylcellulose calcium, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, medium chain triglycerides, methylcellulose, polyoxyethylene sorbitan fatty acid esters, polyvinylpyrrolidinone, propylene glycol alginate, sodium alginate, sorbitan fatty acid esters, and tragacanth. Non-limiting examples of dispersing agents include poloxamer, polyoxyethylene sorbitan fatty acid esters and sorbitan fatty acid esters.

In certain embodiments, the core further comprises at least one disintegrant selected from the group consisting of croscarmellose sodium, crospovidone (cross-linked PVP), sodium carboxymethyl starch (sodium starch glycolate), cross-linked sodium carboxymethyl cellulose (Croscarmellose), pregelatinized starch (starch 1500), microcrystalline starch, water insoluble starch, calcium carboxymethyl cellulose, and magnesium aluminum silicate, or a combination thereof. In other embodiments, the core further comprises at least one of an absorption enhancer, a binder, a hardness enhancing agent, a buffering agent, a filler, a flow regulating agent, a lubricant, a synergistic agent, a chelator, an antioxidant, a stabilizer and a preservative. Optionally, the core also comprises one or more other excipients.

In one embodiment, the outer layer comprises at least one swellable polymer. Non-limiting examples of swellable polymers for use in a controlled release formulation of the invention include acrylic copolymers, e.g., EUDRAGIT RL, EUDRAGIT RS, or EUDRAGIT NE; polyvinylacetate, e.g., KOLLICOAT SR 30D; and cellulose derivatives such as ethylcellulose or cellulose acetate, e.g., SURELEASE and AQUACOAT ECD. Further non-limiting examples of swellable polymers that can be used in the sustained release formulations of the invention include poly(hydroxalkyl methacrylate) having a molecular weight of from 30,000 to 5,000.000; kappa-carrageenan; polyvinylpyrrolidone having a molecular weight of from 10,000 to 360,000; anionic and cationic hydrogels; polyelectrolyte complexes; poly(vinyl alcohol) having low amounts of acetate, cross-linked with glyoxal, formaldehyde, or glutaraldehyde and having a degree of polymerization from 200 to 30,000; a mixture comprising methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; polysaccharide, water swellable gums, high viscosity hydroxylpropylmethyl cellulose and/or mixtures thereof. In certain embodiments, the swellable polymer is selected from the group consisting of calcium pectinate, cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen, modified cellulose, and cross-linked polyacrylic acid. Non-limiting examples of a cross-linked polysaccharide include insoluble metal salts or cross-linked derivatives of alginate, pectin, xantham gum, guar gum, tragacanth gum, and locust bean gum, carrageenan, metal salts thereof, and covalently cross-linked derivatives thereof. Non-limiting examples of modified cellulose include cross-linked derivatives of hydroxypropylcellulose, hydroxypropylmethylcellulose, hydroxyethylcellulose, methylcellulose, carboxymethylcellulose, and metal salts of carboxymethylcellulose.

In another embodiment, the outer layer comprises a water insoluble polymer and a pore-forming agent. Non-limiting examples of pore forming agents include saccharose, sodium chloride, potassium chloride, polyvinylpyrrolidone, and/or polyethyleneglycol, water soluble organic acids, sugars and sugar alcohol. In certain embodiments, the pore forming agent forms part of an outer layer or coating. In other embodiments, the pore forming agent is distributed uniformly throughout the water insoluble polymer. Non-limiting examples of water insoluble polymers include a dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonia Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes. In certain embodiments, the water insoluble particulate matter is a hydrophilic yet water insoluble polymer, preferably selected from the group consisting of a water insoluble cross-linked polysaccharide, a water insoluble cross-linked protein, a water insoluble cross-linked peptide, water insoluble cross-linked gelatin, water insoluble cross-linked hydrolyzed gelatin, water insoluble cross-linked collagen, water insoluble cross linked polyacrylic acid, water insoluble cross-linked cellulose derivatives, water insoluble cross-linked polyvinyl pyrrolidone, micro crystalline cellulose, insoluble starch, micro crystalline starch and a combination thereof. Most preferably, the water insoluble particulate matter is microcrystalline cellulose.

In one embodiment, the outer layer comprises a compression coating. Non-limiting examples of materials that can be used as a compression coating include a gum selected from the group consisting of xanthan gum, locust bean gum, galactans, mannans, alginates, gum karaya, pectin, agar, tragacanth, accacia, carrageenan, tragacanth, chitosan, agar, alginic acid, hydrocolloids acacia catechu, salai guggal, indian bodellum, copaiba gum, asafetida, cambi gum, Enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), and cultured plant cell gums including those of the plants of the genera: acacia, actinidia, aptenia, carbobrotus, chickorium, cucumis, glycine, hibiscus, hordeum, letuca, lycopersicon, malus, medicago, mesembryanthemum, oryza, panicum, phalaris, phleum, poliathus, polycarbophil, sida, solarium, trifolium, trigonella, Afzelia africana seed gum, Treculia africana gum, detarium gum, cassia gum, carob gum, Prosopis africana gum, Colocassia esulenta gum, Hakea gibbosa gum, khaya gum, scleroglucan, and zea, as well as mixtures of any of the foregoing.

In one embodiment, the core comprises a burst controlling agent. The burst controlling agent preferably comprises a water insoluble polymer for controlling the rate of penetration of water into the core and raising the internal pressure (osmotic pressure) inside the core. Such a burst controlling agent is preferably able to swell upon contact with liquid. Non-limiting examples of suitable water insoluble polymers include cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid. In one embodiment, the water insoluble polymer is a cross-linked polysaccharide selected from the group consisting of insoluble metal salts or cross-linked derivatives of alginate, pectin, xanthan gum, guar gum, tragacanth gum, and locust bean gum, carrageenan, metal salts thereof, and covalently cross-linked derivatives thereof. In one embodiment, the water insoluble polymer is a modified cellulose selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose. In another embodiment, the water insoluble polymer is selected from calcium pectinate, microcrystalline cellulose, or a combination thereof.

In one embodiment, the outer layer comprises a water insoluble hydrophobic carrier and a pore forming agent comprised of a water insoluble hydrophilic particular matter. The pore forming agent is a water permeable agent which allows entry of liquid into the core. Optionally, the outer layer further comprises at least one of a wetting agent, a suspending agent, a dispersing agent, a stiffening agent, and a plasticizer.

1.2 Administration and Dosing

The MTR agonists for use in the methods of the invention are preferably formulated for oral administration, more preferably formulated as solid dosage forms for oral administration. In certain embodiments, the MTR agonists are formulated for administration by an intradermal, a transdermal, or a subcutaneous route. In one embodiment, the MTR agonists are formulated for intravenous administration. However, the MTR agonists may be formulated for any suitable route of administration, including, by way of example, nasal (e.g., via an aerosol), buccal (e.g., sub-lingual), topical (i.e., both skin and mucosal surfaces, including airway surfaces), intrathecal, intra-articular, intraplural, intracerebral, intra-arterial, intraperitoneal, oral, intralymphatic, intranasal, rectal or vaginal administration, by perfusion through a regional catheter, or by direct intralesional injection.

The formulations of the present invention contain an amount of MTR agonist that is effective for the intended use. Particular dosages are selected based on a number of other factors including the age, sex, species and condition of the patient. Effective amounts can also be extrapolated from dose-response curves derived from in vitro test systems or from animal models.

When administered as an oral dosage form, the dose of the MTR agonist may be measured in units of mg/kg of body weight. Any measurement of dose can be used in conjunction with the compositions and methods of the invention and dosage units can be converted by means standard in the art.

Examples of dosing regimens that can be used in the methods of the invention include, but are not limited to, sustained, daily or intermittently, preferably daily.

Exemplary doses of an MTR agonist include milligram amounts per kilogram of the subject. In one embodiment, the dose is from about 0.02 to 10 mg/kg of body weight or about 0.04 to 5 mg/kg of body weight. In a specific embodiment, the dosage is about 0.05 mg/kg, about 0.1 mg/kg, about 0.5 mg/kg, about 1 mg/kg, about 5 mg/kg, or about 10 mg/kg of the subject's body weight.

In certain embodiments of the methods for treating diabetes, the MTR agonist is administered to the subject at a dose of from about 0.02 to 10 mg/kg of body weight or about 0.04 to 5 mg/kg of body weight of the subject. In particular embodiments, the MTR agonist is administered at a dose of about 0.05 mg/kg, about 0.1 mg/kg, about 0.5 mg/kg, about 1 mg/kg, about 5 mg/kg, or about 10 mg/kg of the subject's body weight. In certain further embodiments, the MTR agonist formulation is administered to the subject on a weekly or biweekly basis. In specific embodiments, a daily dose is at least 0.05 mg, 0.50 mg, 1.0 mg, 5.0 mg, 10 mg, 15 mg, 20 mg, 30 mg, or at least 50 mg.

The dosage regimen utilizing the MTR agonists can be selected in accordance with a variety of factors including type, species, age, weight, sex and the type of disease being treated; the severity (i.e., stage) of the disease to be treated; the route of administration; the renal and hepatic function of the patient; and the particular compound or salt thereof employed. A dosage regimen can be used, for example, to prevent, inhibit (fully or partially), or arrest the progress of the disease.

In accordance with the invention, an MTR agonist can be administered by continuous/sustained or intermittent dosages. For example, intermittent administration of an MTR agonist may be administration one to six days per week or it may mean administration in cycles (e.g. daily administration for two to eight consecutive weeks, then a rest period with no administration for up to one week) or it may mean administration on alternate days. The MTR agonist may be administered in cycles, with rest periods in between the cycles (e.g. treatment for two to eight weeks with a rest period of up to a week between treatments). The MTR agonist may be administered for a continuous, sustained period of time ranging anywhere from 1 hour to 24 hours, such as 1 hour, 2 hours, 3 hours, 4 hours 5 hours, 6 hours, 7 hours, 8 hours, 9 hours, 10 hours, 11 hours, 12 hours, up to 24 hours, and including half-hour increments (e.g., 1.5 hours, 2.5 hours and the like). Preferably, the MTR agonist can be administered as a sustained release formulation that administers the MTR agonist for a continuous period of time of up to about 12 hours, i.e., 1 hour, 2 hours, 3 hours, 4 hours 5 hours, 6 hours, 7 hours, 8 hours, 9 hours, 10 hours, 11 hours, and 12 hours, more preferably 6 hours to 12 hours.

For example, the MTR agonist can be administered in a total daily dose that is at least 0.05 mg, 0.50 mg, 1.0 mg, 2.0 mg, 3.0 mg, 4.0 mg, 5.0 mg, 10 mg, 15 mg, 20 mg, 30 mg, or at least 50 mg. The MTR agonist can be administered once daily (QD), or divided into multiple daily doses such as twice daily (BID), and three times daily (TID).

In one embodiment, the composition is administered once daily at a dose of about 1.0 mg to about 5.0 mg for a continuous period of 6 hours. In another embodiment, the composition is administered once daily at a sustained dose of about 1.0 mg to about 5.0 mg for a continuous period of up to about 12 hours (i.e., 1 hour to 12 hours or more). In another embodiment, the composition is administered twice daily at a dose of, e.g., about 0.5 mg (or any dosage between 0.05 mg and 50 mg) for a continuous period of 6 hours. Alternatively, the composition can be administered once or twice daily at a dose of, for example, 0.5 or 1 mg (or at any dosage between 0.05 mg and 50 mg) intermittently, for example two, three, four or five days per week.

The MTR agonist can be administered in accordance with any dose and dosing schedule that, together with the effect of one or more therapeutic agents, achieves a dose effective to treat type 2 diabetes. The MTR agonist can be administered in a total daily dose that may vary from patient to patient, and may be administered at varying dosage schedules.

A particular treatment protocol comprises continuous, sustained administration for 6 to 12 hours (i.e., every day), once or twice daily at a total daily dose in the range of about 0.05 mg to 50 mg. This can be achieved by, e.g., a controlled release or sustained release formulation of the MTR agonist. Another treatment protocol comprises intermittent administration of between two to five days a week for 6 to 12 hours, once or twice daily at a total daily dose in the range of about 0.05 mg to 50 mg.

It is apparent to a person skilled in the art that any one or more of the specific dosages and dosage schedules of the MTR agonists are also applicable to any one or more of the therapeutic agents to be used in the combination treatment. Moreover, the specific dosage and dosage schedule of the therapeutic agent can further vary, and the optimal dose, dosing schedule, and route of administration can be determined based upon the specific therapeutic agent that is being used. Further, the various modes of administration, dosages, and dosing schedules described herein merely set forth specific embodiments and should not be construed as limiting the broad scope of the invention. Any permutations, variations, and combinations of the dosages and dosing schedules are included within the scope of the present invention.

1.3 Combination Therapy

According to the present invention, an MTR agonist can be administered in combination with one or more additional therapeutic agents. In one embodiment, the MTR agonist is administered in combination with one or more additional therapeutic agents selected from one or more of the following classes of compounds: insulin secretagogues such as sulfonylureas and meglitinides, biguanides, insulin sensitizers such as thiazolidinediones, DPP-4 inhibitors, alpha-glucosidase inhibitors, incretins and incretin analogues, and hypolipidemic agents.

With respect to the dosage of the one or more additional therapeutic agents, there is no fixed dosage regimen for the management of hyperglycemia in patients with type 2 diabetes with a pharmacologic agent. Dosage must be individualized on the basis of both effectiveness and tolerance, while not exceeding the maximum recommended daily doses.

The route of administration of the MTR agonist is independent of the route of administration of the one or more additional therapeutic agents. A particular route of administration for the MTR agonist is oral administration. Thus, in accordance with this embodiment, the MTR agonist is administered orally, the second, and optionally third and/or fourth anti-diabetic agent can be administered by any suitable route, including, by way of example, nasal (e.g., via an aerosol), buccal (e.g., sub-lingual), topical (i.e., both skin and mucosal surfaces, including airway surfaces), intrathecal, intra-articular, intraplural, intracerebral, intra-arterial, intraperitoneal, oral, intralymphatic, intranasal, rectal or vaginal administration, by perfusion through a regional catheter, or by direct intralesional injection.

Either the MTR agonist or the one or more additional therapeutic agents, or both, may be present as a controlled release or sustained release formulation (which can include, e.g., delayed release formulations). In this regard, either the MTR agonist or the one or more additional therapeutic agents, or both, may be present as an immediate release formulation. In one embodiment, the invention concerns a pharmaceutical composition comprising an MTR agonist and one or more additional therapeutic agents for the treatment of diabetes, wherein the composition is formulated as a controlled release formulation to provide for the immediate release of the MTR agonist and the delayed release of the one or more additional therapeutic agents. In another embodiment, the invention concerns a pharmaceutical composition comprising an MTR agonist and one or more additional therapeutic agents for the treatment of diabetes, wherein the composition is formulated as a controlled release formulation to provide for the sustained release of the MTR agonist and the immediate release of the one or more additional therapeutic agents. The invention also embraces pharmaceutical compositions comprising an MTR agonist and one or more therapeutic agents formulated as an immediate release formulation, or where both the MTR agonist and the one or more therapeutic agents are formulated for delayed release. In some embodiments, the MTR agonist is administered as a sustained release formulation for up to about 12 hours, preferably 6 to 12 hours, and the one or more additional therapeutic agents is administered for delayed release before a meal and after the sustained 6 to 12 hour release of the MTR agonist. Without wishing to be bound by theory, the type or class of therapeutic agent will determine if delayed or immediate release is preferable. For example, combination therapy with the MTR agonist and, e.g., an insulin secretagogue or a dipeptidyl peptidase 4 inhibitor may be preferable when the insulin secretagogue or dipeptidyl peptidase 4 inhibitor is administered for delayed release before a meal and after the 6 to 12 hour sustained release of the MTR agonist. In this regard, combination therapy with the MTR agonist and, e.g., a thiazolidinedione or a biguanide such as metformin does not expressly depend upon the delayed or immediate release of the thiazolidinedione or the biguanide and thus could be for delayed or immediate release.

1.3.1 Insulin Secretagogues

In one embodiment, the methods of the present invention comprise the administration of an MTR agonist in combination with one or more insulin secretagogues, agents that stimulate insulin release from pancreatic beta cells. In a particular embodiment, the one or more secretagogues is selected from a sulfonylurea or a meglitinide. In one embodiment, the sulfonylurea is selected from the group consisting of chlorpropamide (DIABINESE), glipizide (GLUCOTROL, GLUCOTROL XL), glyburide (MICRONASE, GLYNASE, DIABETA), and glimepiride (AMARYL). In one embodiment, the meglitinide is selected from the group consisting of repaglinide (PRANDIN) and nateglinide (STARLIX).

In one embodiment, the method comprises administration of the MTR agonist 2 to 12 hours before a meal (which administration is sustained far a period of time of up to about 12 hours, preferably about 6 hours to about 12 hours) and administration of the one or more secretagogues within 1 hour of the meal or with the meal. In another embodiment, the MTR agonist is formulated as a controlled release formulation with the one or more secretagogues such that the MTR agonist is released within 1 to 2 hours following administration, which administration is sustained for a period of time of up to about 12 hours, preferably about 6 hours to about 12 hours and the one or more secretagogues is released about 2, 4, 6, 8, 10, or 12 hours after the initiation of release of the MTR agonist.

The dosage of the secretagogue is determined according to standard protocols, for example, by monitoring the patient's blood glucose levels to determine the minimum effective dose, to detect primary failure, i.e., inadequate lowering of blood glucose at the maximum recommended dose of medication; and to detect secondary failure, i.e., loss of an adequate blood glucose lowering response after an initial period of effectiveness. Glycosylated hemoglobin levels may also be of value in monitoring the patient's response to therapy.

In a preferred embodiment, the total daily dosage of MTR agonist and the one or more secretagogues is given in a single daily administration of each, or in a single daily administration containing both where the MTR agonist and the secretagogue(s) are formulated as a controlled release formulation.

1.3.2 Biguanides

In one embodiment, the methods of the present invention comprise the administration of an MTR agonist in combination with one or more biguanides. Biguanides lower blood glucose levels primarily by decreasing the amount of glucose produced by the liver via gluconeogenesis. Some biguanides also help to lower blood glucose levels by making muscle tissue more sensitive to insulin. Examples of biguanides include, but are not limited to, metformin (also known in the art as GLUCOPHAGE). In one embodiment, the MTR agonist is administered in combination with metformin (GLUCOPHAGE). Preferably, the MTR agonist is administered prior to the metformin. Metformin may be administered once or twice daily or in any increment to achieve the desired steady-state levels in the bloodstream. In certain embodiments, the MTR agonist is administered 2, 4, 6, 8, 10, or 12 hours prior to the metformin, which is taken with a meal. In another embodiment, the MTR agonist and the metformin are formulated in a controlled release formulation such that the MTR agonist is released within 1 to 2 hours following administration, which administration is sustained for a period of time of up to about 12 hours, preferably about 6 hours to about 12 hours, and the metformin is released about 2, 4, 6, 8, 10, or 12 hours after the initiation of release of the MTR agonist. The timing of metformin release can coincide with the taking of a meal and may be released after the sustained release of the MTR agonist, i.e. sustained release for 6 to 12 hours. Alternatively, the timing of metformin release can be immediate and not dependent upon the taking of a meal or the release of the MTR agonist.

1.3.3 Thiazolidinediones

In one embodiment, the methods of the present invention comprise the administration of an MTR agonist in combination with one or more thiazolidinediones or related insulin sensitizers and/or compounds that modulate PPAR gamma. Thiazolidinediones reduce glucose production in the liver and potentiate the action of insulin in muscle and fat. In one embodiment, the one or more thiazolidinediones is selected from the group consisting of rosiglitazone (AVANDIA) and pioglitazone (ACTOS).

Preferably, the MTR agonist is administered prior to the one or more thiazolidinediones. Thiazolidinediones may be administered once or twice daily or in any increment sufficient to achieve the desired steady-state levels in the bloodstream of the subject. In certain embodiments, the MTR agonist is administered 2, 4, 6, 8, 10, or 12 hours prior to the one or more thiazolidinediones, which is taken with a meal (and wherein the MTR agonist administration is sustained for a period of time of up to about 12 hours, preferably about 6 hours to about 12 hours. In another embodiment, the MTR agonist and the one or more thiazolidinediones are formulated in a controlled release formulation such that the MTR agonist is released within 1 to 2 hours following administration, which administration is sustained for a period of time of up to about 12 hours, preferably about 6 hours to about 12 hours, and the one or more thiazolidinediones is released about 2, 4, 6, 8, 10, or 12 hours after the initiation of release of the MTR agonist. The timing of thiazolidinedione release can coincide with the taking of a meal and may be released after the sustained release of the MTR agonist, i.e. sustained release for 6 to 12 hours. Alternatively, the timing of thiazolidinedione release can be immediate and not dependent upon the taking of a meal or the release of the MTR agonist.

In one embodiment, the MTR agonist is administered in combination with one or more thiazolidinediones and a sulfonylurea, metformin, or insulin.

1.3.4 Dipeptidyl Peptidase 4 Inhibitors

In one embodiment, the methods of the present invention comprise the administration of an MTR agonist in combination with one or more dipeptidyl peptidase 4 (DPP4) inhibitors. DPP4 cleaves and inactivates the incretin hormone GLP-1. Thus, DPP-4 inhibitors work by inhibiting the degradation of GLP-1. In one embodiment, the DPP4 inhibitor is sitagliptin (JANUVIA), but can also include saxagliptin (ONGLYZA).

Preferably, the MTR agonist is administered prior to the one or more DPP-4 inhibitors. In certain embodiments, the MTR agonist is administered 2, 4, 6, 8, 10, or 12 hours prior to the one or more DPP-4 inhibitors. and the one or more DPP-4 inhibitors is released about 2, 4, 6, 8, 10, or 12 hours after the release of the MTR agonist. The MTR agonist can be administered prior to the taking of a meal. In certain embodiments, the MTR agonist is administered 2, 4, 6, 8, 10, or 12 hours prior to the meal, which administration is sustained for a period of time of up to about 12 hours, preferably about 6 hours to about 12 hours. The one or more DPP4 inhibitors can be taken with or without food.

Preferably, In one embodiment, the MTR agonist and the one or more DPP4 inhibitors are formulated for immediate release for a sustained period of time, continuously for a period of time up to 12 hours, preferably 6 to 12 hours and the one or more DPP4 inhibitors is released at any time after the initiation of release of the MTR agonist. In another embodiment, the MTR agonist and the one or more DPP-4 inhibitors are formulated in a controlled release formulation such that the MTR agonist is released within 1 to 2 hours following administration and which administration is sustained for a period of time of up to about 12 hours, preferably about 6 hours to about 12 hours, and the one or more DPP4 inhibitors is released at any time after the initiation of release of the MTR agonist.

In one embodiment, the MTR agonist and the one or more DPP4 inhibitors are administered as part of a regimen that includes diet and exercise to improve glycemic control in patients with type 2 diabetes. In one embodiment, the MTR agonist and the one or more DPP4 inhibitors are administered in combination with a sulfonylurea. In a particular embodiment, the sulfonylurea is selected from the group consisting of chlorpropamide (DIABINESE), glipizide (GLUCOTROL, GLUCOTROL XL), glyburide (MICRONASE, GLYNASE, DIABETA), and glimepiride (AMARYL).

1.3.5 Alpha-Glucosidase Inhibitors

In one embodiment, the methods of the present invention comprise the administration of an MTR agonist in combination with one or more alpha-glucosidase inhibitors. Alpha-glucosidase inhibitors lower blood glucose levels and attenuate the rise in blood glucose levels after a meal by blocking the breakdown of starches and, slowing the breakdown of some sugars. In one embodiment, the one or more alpha-glucosidase inhibitors is selected from the group consisting of acarbose (PRECOSE) and meglitol (GLYSET).

Preferably, the MTR agonist is administered prior to the one or more alpha-glucosidase inhibitors. In certain embodiments, the MTR agonist is administered 2, 4, 6, 8, 10, or 12 hours prior to the one or more alpha-glucosidase inhibitors, which should be taken with the first bite of a meal, wherein the MTR agonist administration is sustained for a period of time of up to about 12 hours, preferably about 6 hours to about 12 hours. In another embodiment, the MTR agonist and the one or more alpha-glucosidase inhibitors are formulated in a controlled release formulation such that the MTR agonist is released within 1 hour following administration, which administration is sustained for a period of time of up to about 12 hours, preferably about 6 hours to about 12 hours and the one or more alpha-glucosidase inhibitors is released about 2, 4, 6, 8, 10, or 12 hours after the initiation of release of the MTR agonist. Preferably, the timing of alpha-glucosidase inhibitor release coincides with the taking of a meal.

1.3.6 GLP-1 Receptor Agonists

In one embodiment, the methods of the present invention comprise the administration of an MTR agonist in combination with one or more activators of the GLP-1 receptor. GLP-1 is an incretin hormone. Incretins are gut-associated peptide hormones that enhance glucose-dependent insulin secretion and exhibit other antihyperglycemic actions following their release into the circulation from the gut following a meal. In one embodiment, the one or more agonists of the GLP-1 receptor is selected from GLP-1, exendin-4, VICTOZA and BYETTA (exenatide).

Exendin-4 is a naturally occurring peptide agonist of the GLP-1 receptor originally isolated from the venom of the Heloderma suspectum lizard. It is more resistant to cleavage by DPP-4 and thus has a longer circulating half-life than GLP-1. BYETTA (exenatide) is an incretin mimetic and a synthetic version of exendin-4. It mimics the enhancement of glucose-dependent insulin secretion and several other antihyperglycemic actions of incretins. For example, BYETTA enhances glucose-dependent insulin secretion by the pancreatic beta-cell, suppresses inappropriately elevated glucagon secretion, and slows gastric emptying.

Preferably, the MTR agonist is administered prior to the one or more agonists of the GLP-1 receptor. In certain embodiments, the MTR agonist is administered 2, 4, 6, 8, 10, or 12 hours prior to the one or more agonists of the GLP-1 receptor, which should be about 1 hour prior to a meal, wherein the MTR agonist administration is sustained for a period of time of up to about 12 hours, preferably about 6 hours to about 12 hours. In another embodiment, the MTR agonist and the one or more agonists of the GLP-1 receptor are formulated in a controlled release formulation such that the MTR agonist is released within 1 to 2 hours following administration, which administration is sustained for a period of time of up to about 12 hours, preferably about 6 hours to about 12 hours and the one or more agonists of the GLP-1 receptor is released about 2, 4, 6, 8, 10, or 12 hours after the initiation of release of the MTR agonist. Preferably, the timing of GLP-1 receptor agonist release occurs within about an hour before the taking of a meal.

In one embodiment, the MTR agonist and the one or more GLP-1 receptor agonists are administered as part of a regimen to improve glycemic control in patients with type 2 diabetes. In one embodiment, the regimen includes metformin, a sulfonylurea, a thiazolidinedione, a combination of metformin and a sulfonylurea, or a combination of metformin and a thiazolidinedione.

1.3.7 Fenofibrate Containing Hypolipidemic Agents

In one embodiment, the methods of the present invention comprise the administration of an MTR agonist in combination with one or more hypolipidemic agents. In a specific embodiment, the one or more hypolipidemic agents is a fenofibrate containing hypolipidemic agent. In one embodiment, the one or more hypolipidemic agents is selected from LIPOFEN and TRIGLIDE.

Preferably, the MTR agonist is administered prior to the one or more hypolipidemic agents. The one or more hypolipidemic agents may be administered once or twice daily or in any increment sufficient to achieve the desired steady-state levels in the bloodstream of the subject. In certain embodiments, the MTR agonist is administered 2, 4, 6, 8, 10, or 12 hours prior to the one or more hypolipidemic agents, wherein the MTR agonist administration is sustained for a period of time of up to about 12 hours, preferably about 6 hours to about 12 hours. In one embodiment, the hypolipidemic agent is taken with food. In another embodiment, the hypolipidemic agent is taken without food. In one embodiment, the MTR agonist and the one or more hypolipidemic agents are formulated in a controlled release formulation such that the MTR agonist is released within 1 hour following administration, which administration is sustained for a period of time of up to about 12 hours, preferably about 6 hours to about 12 hours and the one or more hypolipidemic agents is released about 2, 4, 6, 8, 10, or 12 hours after the initiation of release of the MTR agonist. In one embodiment, the timing of the release of the hypolipidemic agent coincides with the taking of a meal and may be released after the sustained release of the MTR agonist, i.e. sustained release for 6 to 12 hours. Alternatively, the timing of hypolipidemic agent release can be immediate and not dependent upon the taking of a meal or the release of the MTR agonist.

In one embodiment, the MTR agonist and the one or more hypolipidemic agents are administered as part of a regimen to improve glycemic control in patients with type 2 diabetes. In a specific embodiment, the regimen includes an appropriate lipid-lowering diet.

The invention is illustrated in the examples that follow. This section is set forth to aid in an understanding of the invention but is not intended to, and should not be construed to limit in any way the invention as set forth in the claims which follow thereafter.

### EXAMPLES

### Example 1

### Effect of 12-Hour Sustained Release of Melatonin on Glucose and Insulin Levels in Mice

In this experiment, male lean C57BL6 mice were used. The animals were subjected to specifically timed light cycles, wherein the “light” period and the “dark” period start at 5:00 AM and 5:00 PM, respectively. “Time 0 on Day 0” is 5:00 am of the test day. Details of each group of mice and the regimen received is summarized in Table 1 below.

TABLE 1
Study Design

| Group # | N | Test Article | Regimen | End Points |
|---|---|---|---|---|
| 1 | 9 | Vehicle (oil) | SC, −12 h | 1. Insulin at Time 0, 30, 60, and |
| 2 | 9 | Melatonin | SC, −12 h | 120 minutes. |
| 3 | 5 | Melatonin | SC, −1 h | 2. Oral glucose test, Time 0, 5, |
| 4 | 4 | Vehicle (oil) | SC, −1 h | 10, 20, 30, 60, and 120 minutes. |

“SC” = subcutaneous administration

The mice were randomized to achieve similar body weight and glucose for each group before dosing. All animals were subjected to fasting at the start of the dark period (Group 1-4). Food was removed, and bedding materials were replaced with fresh bedding to eliminate any residual food. Animals remained under fast until the 120 minute time point was taken. Water was made available ad libitum throughout the study.

A 30 mg/mL stock solution of melatonin (30 mg melatonin in 1 ml of pure sesame oil) was made. From this stock solution, 10 μL of stock was taken and diluted with 990 μL of sesame oil, resulting in a solution concentration of 300 μg/ml. This was then diluted 1:18, resulting in a final concentration of 16.6-16.7 μg/mL. Mice received 0.3 ml by subcutaneous injection (5 μg/mouse). Injections were performed in the dark under infrared light.

Insulin and blood glucose levels were measured by standard methods. The oral glucose tolerance test was carried out as follows using a glucose oxidase reaction measured by a Precision glucometer. Oral gavage of 3 g/kg glucose was administered to all animals at Day 0, Time 0. A 1 mm tip of the tail was cut for the first time point, and an aliquot of 5-10 μL of blood was obtained from the tail tip cut and applied to the glucose test strip of a glucose meter. For subsequent time points, the first drop of the blood from the open wound was discarded, and the second drop applied to the glucose test strip. Blood glucose level of all animals in Group 1-4 were measured at time 0 (right before the glucose dosing), 5, 10, 20, 30, 60 and 120 minutes post glucose dosing (7 time points). Insulin levels were also measured at 0, 30, 60, and 120 minutes from separate collections of tail vein blood obtained post glucose dosing by Crystal Chemistry ELISA.

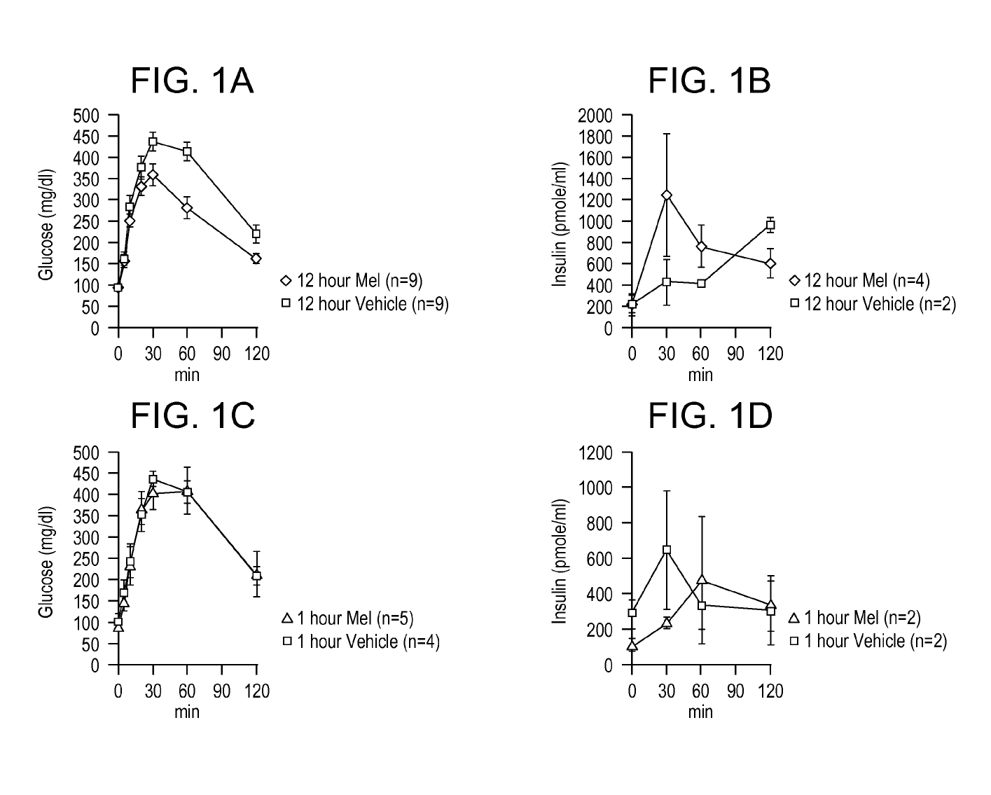

FIG. 1 shows a comparison of results after mice were given a dose of melatonin that was sustained over a 12 hour period of time (the “12-hour melatonin dose”), a dose of melatonin that was sustained over a 1 hour period of time (the “1-hour melatonin dose”), or vehicle alone. FIG. 1A depicts a graph showing that the blood glucose levels of mice receiving the 12-hour sustained melatonin dose were significantly decreased compared to the blood glucose levels of mice receiving vehicle alone. In FIG. 1B, the graph shows that the insulin levels of mice receiving the 12-hour melatonin dose was much higher than mice receiving vehicle alone at the same time point. The data here tend to show that a 12-hour sustained dose of melatonin produced a significant effect on blood glucose and insulin levels compared to control.

Importantly, a dose of melatonin delivered for a sustained period of 1 hour prior to glucose administration did not produce the same effect as the 12-hour melatonin dose. FIG. 1C shows that mice receiving the 1-hour melatonin dose did not experience the same reduction in post-challenge glucose excursion as the mice receiving the 12-hour melatonin dose, and that the glucose levels here are not appreciably different from the levels measured in mice receiving vehicle alone. Similarly, mice receiving the 1-hour melatonin dose did not experience the same augmentation of glucose-stimulated insulin secretion as the mice receiving the 12-hour melatonin dose did. See FIG. 1D. Interestingly, insulin levels were lower.

In summary, the results of this experiment show an unexpected and surprising effect of a 12-hour sustained melatonin dose on blood glucose and insulin levels. This effect appears to be unique to the 12-hour melatonin dose, since mice receiving a 1-hour melatonin dose did not have the same drop in blood glucose or the same increase in insulin levels.

### Example 2

### Effect of a Combination of Melatonin and the DPP IV Inhibitor Sitagliptin on Glucose and Insulin Levels in Mice

Based on the results obtained in the preceding experiment, the 12-hour sustained melatonin dose was tested with a known anti-diabetic drug, sitagliptin. Sitagliptin is a dipeptidyl peptidase IV inhibitor marketed as “JANUVIA®”.

In this experiment, male C57BL6 DIO mice (10-11 weeks old, n=52) were used and maintained on a high fat diet. A total of 49 animals were used in this study. The animals were subjected to specifically timed light cycles, wherein the “light” period and the “dark” period start at 8:00 AM and 8:00 PM, respectively. “Time 0 on Day 0” is 8:00 am of the test day. Details of each group of mice and the regimen received is summarized in Table 2 below.

TABLE 2
Study Design

| Group # | N | Test Article | Regimen | End Points |
|---|---|---|---|---|
| 1 | 7 | Vehicle (oil) | SC, −12 h | 1. Body weight at Time 0 |
| 2 | 7 | Oral vehicle | PO, Day 0 −1 h | 2. Oral glucose test, Time 0, 15, |
| 3 | 7 | Oil and oral vehicle | SC, −12 h | 30, 60, and 120 minutes. |
| | | | PO, Day 0 −1 h | |
| 4 | 7 | Melatonin alone | SC, −12 h | |
| 5 | 7 | Sitagliptin alone | PO, Day 0 −1 h | |
| 6 | 7 | Melatonin & Sitagliptin | SC, −12 h | |
| | | | PO, Day 0 −1 h | |
| 7 | 7 | No treatment | N/A | |

“SC” = subcutaneous administration
“PO” = oral administration

The mice were randomized to achieve similar body weight and glucose for each group before dosing. All animals were subjected to fasting at the start of the dark period (Groups 1-7). Food was removed, and bedding materials were replaced with fresh bedding to eliminate any residual food. Animals remained under fast until the 120 minute time point was taken. Water was made available ad libitum throughout the study.

A 30 mg/mL stock solution of melatonin (30 mg melatonin in 1 ml of pure sesame oil) was made. From this stock solution, 10 μL of stock was taken and diluted with 990 μL of sesame oil, resulting in a solution concentration of 300 μg/ml. This was then diluted 1:18, resulting in a final concentration of 16.6-16.7 μg/mL. The doses of melatonin were adjusted based on the weight of the mouse. For example, for a 25 g mouse, 5 μg of melatonin was administered; for a 30 g mouse, 6 μg of melatonin was administered, or 300 μL of 20 μg/mL. The total dose volume of melatonin and the vehicle was 300 μL/30 g mouse, or 10 mL/kg. The final concentration for injection was 20 μg/mL, or 6 μg/300 μL). The vehicle was prepared by making aliquots of 7 mL of sesame oil (Pure sesame oil, Kadoya Sesame Mills, Inc., Tokyo, Japan). Melatonin and vehicle (Groups 1 and 4) were dosed once at −12 h, subcutaneously.

Sitagliptin was prepared in an oral dosing vehicle comprising polyethylene glycol (PEG)-400 and Tween™-80. The oral dosing vehicle was prepared by mixing 9.95 mL of PEG-400 with 0.05 mL of Tween-80. The final ratio of PEG400:Tween-80 was 99.5:0.05. Carboxymethylcellulose (CMC) was then added (1.809 mL of 0.5% CMC) to 191 μL of the PEG400-Tween mixture to reach a final concentration of PEG400 to 9.5%. Sitagliptin was added to this mixture to result in a final dose of 10 mg/kg. The dosing volume of sitagliptin and the oral dosing vehicle was 100 μL/30 grams, or 3.3 mL/kg. The final concentration for oral dosing was 3 mg/mL. Sitagliptin and oral dosing vehicle (Groups 2, 3, 5, and 6) were administered orally on Day 0, at −1 h before glucose administration. A similar experiment was performed using sitagliptin at a final dose of 3 mg/kg.

Blood glucose levels were measured by standard methods. The oral glucose tolerance test was carried out as follows. Oral gavage of 3 g/kg glucose was administered to all animals at Day 0, Time 0, A 1 mm tip of the tail was cut for the first time point, and an aliquot of 5-10 mL of blood was be obtained from the tail tip cut and applied to the glucose test strip of a glucose meter. For subsequent time points, the first drop of the blood from the open wound was discarded, and the second drop applied to the glucose test strip. Blood glucose level of all animals in Groups 1-7 were measured at time 0 (right before the glucose dosing), 15, 30, 60 and 120 minutes post glucose dosing (5 time points). Body weight was recorded twice per week prior to the study, and before the blood glucose level measurements. Average body weight was used for glucose administration.

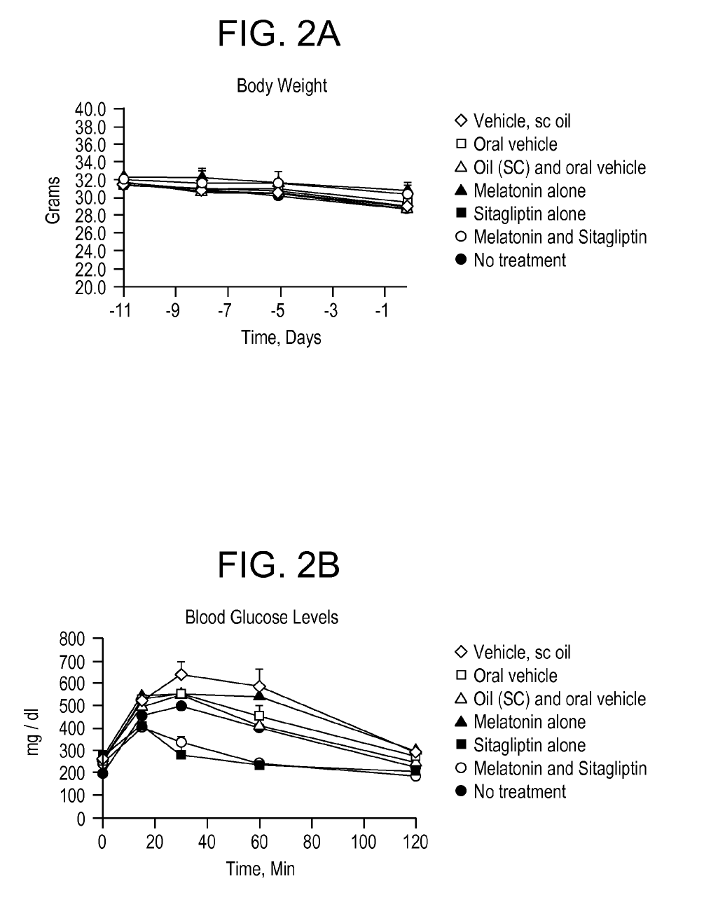

FIG. 2 shows a comparison of results of the 12 hour melatonin dose alone, sitagliptin administration alone, and the 12 hour melatonin dose and sitagliptin together. While Groups 1-7 did not show an appreciable difference in body weight (see FIG. 2A), it is notable that the combination of melatonin and sitagliptin showed a marked decrease in blood glucose levels (see FIG. 2B). Sitagliptin (10 mg/kg) alone, as expected, resulted in a decrease in blood glucose levels. While this experiment showed an unexplained effect of the oil in the formulation on blood glucose levels, however relative to the animals receiving no treatment, those receiving the combination of the 12 hour dose of melatonin and sitagliptin did show a decrease in blood glucose levels. Importantly, this experiment demonstrated that, contrary to prior studies from other investigators, melatonin does not inhibit the anti-diabetic action of known diabetes drugs, such as sitagliptin. Similar results were found when sitagliptin was administered at a lower dose (3 mg/kg; data not shown).

In summary, the experiments show that there is a kinetic basis for an anti-diabetic action of melatonin. Previous work implicated melatonin as a possible mixed agonist/antagonist of the clinical identifiers of type 2 diabetes (such as blood glucose levels and insulin secretion stimulated by glucose). Notably, a 1-hour dose of melatonin did not affect glucose excursion or insulin secretion compared to control, but a 12-hour sustained dose of melatonin did. In contrast to prior studies, a 12-hour sustained dose of melatonin does not antagonize the anti-diabetic effect of sitagliptin, suggesting that melatonin administration can be modulated to take specific advantage of its anti-diabetic activity as an agonist while eliminating or mitigating its activity as an antagonist.

### EQUIVALENTS

Those skilled in the art will recognize or be able to ascertain using no more than routine experimentation, many equivalents to the specific embodiments of the invention described herein. Such equivalents are intended to be encompassed by the following claims. All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A method for the treatment of type 2 diabetes in a human subject in need thereof, comprising administering to the subject an effective amount of a melatonin receptor (MTR) agonist about 6-12 hours before a meal.

2. The method of claim 1, wherein the MTR agonist is melatonin.

3. The method of claim 1, wherein the MTR agonist is administered in combination with one or more additional therapeutic agents.

4. The method of claim 3, wherein the one or more additional therapeutic agents is selected from the group consisting of a biguanide, a glucagon-like peptide 1 receptor activator, a dipeptidyl peptidase 4 inhibitor, an insulin sensitizer, and a hypolipidemic agent.

5. The method of claim 3, wherein the one or more additional therapeutic agents is selected from the group consisting of a dipeptidyl peptidase 4 inhibitor, an insulin sensitizer, and a hypolipidemic agent.

6. The method of claim 3, wherein the one or more additional therapeutic agents is selected from the group consisting of an activator of the gastric inhibitory peptide receptor, a glucokinase activator, a ghrelin receptor agonist, an orexin receptor agonist, an oxyntomodulin receptor agonist, a G-protein coupled receptor 40 or 119 agonist, glucose-dependent insulinotropic peptide, an agonist of an RGS protein, and an insulin secretagogue.

7. The method of claim 1, wherein the MTR agonist is formulated for oral administration.

8. The method of claim 1, wherein the MTR agonist is formulated as a sustained release formulation.

9. The method of claim 8, wherein the sustained release formulation administers the MTR agonist for a period of time of up to about 12 hours.

10. The method of claim 9, wherein the sustained release formulation administers the MTR agonist for about 6 hours to about 12 hours.

11. The method of claim 1, wherein the MTR agonist is a molecule that crosses the blood brain barrier or is formulated to cross the blood brain barrier.

12. The method of claim 1, wherein the MTR agonist is a molecule that does not cross the blood brain barrier or is formulated not to cross the blood brain barrier.

13. The method of claim 3, wherein the one or more additional therapeutic agents does not include an antioxidant or zinc.

14. The method of claim 3, wherein the one or more additional therapeutic agents does not include an insulin secretagogue, a biguanide, GLP-1, or a GLP-1 receptor activator.

15. The method of claim 3, wherein the one or more additional therapeutic agents comprises a dipeptidyl peptidase 4 inhibitor.

16. The method of claim 15, wherein the dipeptidyl peptidase 4 inhibitor is sitagliptin.

17. A pharmaceutical composition comprising a melatonin receptor (MTR) agonist), wherein the composition is formulated as a controlled release formulation to provide for the sustained release of the MTR agonist.

18. The composition of claim 17, wherein the sustained release is for a period of time of up to about 12 hours.

19. The composition of claim 17, wherein the sustained release is for about 6 hours to about 12 hours.

20. A pharmaceutical composition comprising a melatonin receptor (MTR) agonist and one or more additional therapeutic agents for the treatment of diabetes, wherein the composition is formulated as a controlled release formulation to provide for the immediate release of the MTR agonist and the delayed release of the one or more additional therapeutic agents.

21. The composition of claim 20, wherein the release of the one or more additional therapeutic agents is delayed for about 6 to 12 hours.

22. The composition of claim 20, wherein the one or more additional therapeutic agents is selected from glucagon-like peptide 1 (GLP-1), gastric inhibitory peptide, a gastric inhibitory peptide receptor activator, a GLP-1 receptor activator, glucose-dependent insulinotropic peptide, a glucokinase activator, a ghrelin receptor agonist, an orexin receptor agonist, an oxyntomodulin receptor agonist, a G-protein coupled receptor 40 or 119 agonist, an agonist of an RGS protein, a dipeptidyl peptidase 4 inhibitor, a biguanide, an insulin secretagogue, an insulin sensitizer, and a hypolipidemic agent.

23. The composition of claim 20, wherein the one or more additional therapeutic agents does not include an antioxidant or zinc.

24. The composition of claim 20, wherein the one or more additional therapeutic agents does not include an insulin secretagogue, a biguanide, GLP-1, or a GLP-1 receptor activator.

25. The composition of claim 22, wherein the one or more additional therapeutic agents comprises a dipeptidyl peptidase 4 inhibitor.

26. The composition of claim 25, wherein the dipeptidyl peptidase 4 inhibitor is sitagliptin.

27. A pharmaceutical composition comprising a melatonin receptor (MTR) agonist and one or more additional therapeutic agents for the treatment of diabetes, wherein the composition is formulated as a controlled release formulation to provide for the sustained release of the MTR agonist and the immediate release of the one or more additional therapeutic agents.

28. The composition of claim 27, wherein the sustained release of the MTR agonist is for a period of time of up to about 12 hours.

29. The composition of claim 27, wherein the one or more additional therapeutic agents is selected from glucagon-like peptide 1, gastric inhibitory peptide, a gastric inhibitory peptide receptor activator, a GLP-1 receptor activator, glucose-dependent insulinotropic peptide, a glucokinase activator, a ghrelin receptor agonist, an orexin receptor agonist, an oxyntomodulin receptor agonist, a G-protein coupled receptor 40 or 119 agonist, an agonist of an RGS protein, a biguanide, an insulin sensitizer, and a hypolipidemic agent.

30. The composition of claim 27, wherein the one or more additional therapeutic agents does not include an antioxidant or zinc.

31. The composition of claim 27, wherein the one or more additional therapeutic agents does not include an insulin secretagogue, a biguanide, GLP-1, or a GLP-1 receptor activator.

32. A pharmaceutical composition comprising a melatonin receptor (MTR) agonist and one or more additional therapeutic agents for the treatment of diabetes, wherein the composition is formulated as a controlled release formulation to provide for the sustained release of the MTR agonist and the delayed release of the one or more additional therapeutic agents.

33. The composition of claim 32, wherein the release of the one or more additional therapeutic agents is delayed for about 6 to 12 hours.

34. The composition of claim 32, wherein the one or more additional therapeutic agents is selected from glucagon-like peptide 1 (GLP-1), gastric inhibitory peptide, a gastric inhibitory peptide receptor activator, a GLP-1 receptor activator, glucose-dependent insulinotropic peptide, a glucokinase activator, a ghrelin receptor agonist, an orexin receptor agonist, an oxyntomodulin receptor agonist, a G-protein coupled receptor 40 or 119 agonist, an agonist of an RGS protein, a dipeptidyl peptidase 4 inhibitor, a biguanide, an insulin secretagogue, an insulin sensitizer, and a hypolipidemic agent.

35. The composition of claim 32, wherein the one or more additional therapeutic agents does not include an antioxidant or zinc.

36. The composition of claim 34, wherein the one or more additional therapeutic agents does not include an insulin secretagogue, a biguanide, GLP-1, or a GLP-1 receptor activator.

37. The composition of claim 34, wherein the one or more additional therapeutic agents comprises a dipeptidyl peptidase 4 inhibitor.

38. The composition of claim 37, wherein the dipeptidyl peptidase 4 inhibitor is sitagliptin.
